(19) United States
(12) Patent Application Publication
(10) Pub. No.: US 2010/0036493 A1
(43) Pub. Date: Feb. 11, 2010

(54) MAGNETIC CUSHIONING DEVICES

(76) Inventor: Jerome H. Simon, Newton, MA (US)

Correspondence Address: BURNS & LEVINSON, LLP, 125 SUMMER STREET, BOSTON, MA, 02110, US

(21) Appl. No.: 12/536,382

(22) Filed: Aug. 5, 2009

Related U.S. Application Data
(60) Provisional application No. 61/137,940, filed on Aug. 5, 2008.

(51) Int. Cl. A61F 2/08; A61F 2/28

(52) U.S. Cl. 623/14.12; 623/16.11

(57) ABSTRACT

A cushioning device that when implanted provides mobility and wear resisting cushioning to joints within a human body, provides a first component at least in part comprising at least one magnet, a second component at least in part comprising at least one magnet, the magnetic poles of said magnet of said first component, and the magnetic poles of said magnet of said second component, so disposed as to create a repelling force between said first and second components and therefore an opposing movement away from each other, and a third component comprising a magnetically attractable material disposed between said first and second components towards which said corresponding magnets are attracted, said attracting forces balancing and controlling said same repelling force so as to maintain said movement of said first and second components within said predetermined planar degree of freedom.

### CROSS REFERENCE TO RELATED APPLICATIONS

This application claims priority from U.S. Provisional Patent Application Ser. No. 61/137,940, filed Aug. 5, 2008 and incorporated herein by reference in its entirety.

### THE PURPOSE OF THIS INVENTION

To provide a cushioning device that can be implanted on or within moving joints of a living body in order to relieve pain and reduce functional wear between bones of said bony structures.

To provide a mechanical device wherein the magnetic forces create stability between the moving parts of said device.

To simplify the control of magnetic forces by reducing the number of magnets (in some embodiments a single magnet), and, by employing non-magnetic and magnetically attractable materials.

### BRIEF DESCRIPTION OF THE DRAWINGS

FIG. 1—is a three dimensional diagram of a magnetic cushioning device. FIG. 1 describes the motion of the components of the device.

FIG. 2—is a three dimensional diagram of a magnetic cushioning device similar to that of FIG. 1 with a variation in component shape.

FIG. 2A—is a three dimensional diagram of a magnetic cushioning device similar to that of FIG. 2 having a variation in component size.

FIG. 2B—is a side view elevation of FIG. 2A.

FIG. 3—is a side view diagram of a human knee illustrating devices described in FIGS. 2 through 2B as being surgically implanted within the bones of the knee.

FIG. 3A—is a side view diagram of the device shown in FIG. 3, further illustrating movement of the bones.

FIG. 4—is an isometric diagram of a magnetic cushioning device comprising two co-functioning component assemblies.

FIG. 4A—is a cross-sectional diagram of a magnetic cushioning device similar to that illustrated in FIG. 4.

FIG. 4B—Is an isometric diagram of a magnetic cushioning device similar to that of FIG. 4 being acted upon by outside forces aligned along an axis.

FIG. 4C—is a side view of a magnetic cushioning devise as illustrated in FIG. 4B.

FIG. 4D—is a side view of a magnetic cushioning device similar to the one illustrated in 4B said outside forces applied at an angle to each other.

FIG. 4E—is a side view elevation of FIG. 4D with no forces applied

FIG. 5—is an isometric diagram of a magnetic cushioning device similar in function to the one illustrated in FIG. 4.

FIG. 5A—is a cross-sectional diagram of a magnetic cushioning device as illustrated in FIG. 5 with the addition of spacing components.

FIG. 5B—is an isometric diagram of a magnetic cushioning device similar to that illustrated in FIGS. 4 and 4A substituting magnets for non magnet components.

FIG. 5C is an edge view diagram of a magnetic cushioning device as illustrated in FIG. 5B.

### DISCUSSION OF PREFERRED EMBODIMENTS

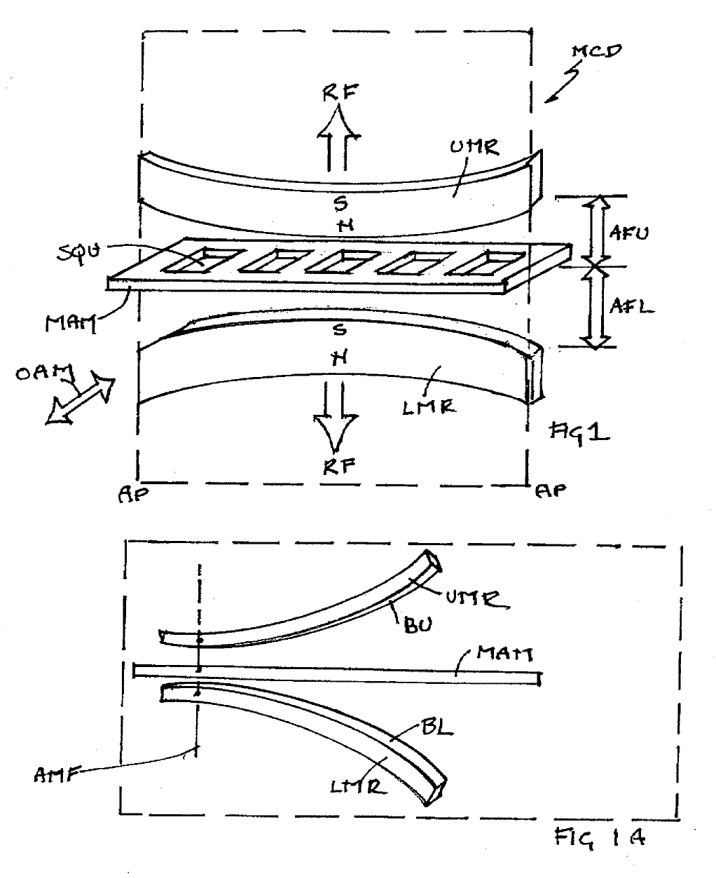

FIG. 1: is a three dimensional diagram of the component system of a magnetic cushioning device MCDC. Two of the three components of MCDC are magnets UMR and LMR and (within this embodiment) are shaped as arcs and are disposed so that similar poles N that are located in proximity to the outside circumference of the arcs face each other so that said magnets UMR and LMR are repelling each other. This repelling force is illustrated by arrows RF/RF. An element of magnetically attractable material MAM (shown in this embodiment) is shaped as a rectangular bar having opening SQU along its length, is disposed between magnets UMR and LMR positioned to maintain the alignment along axial plane AP/AP. This is achieved by balancing the repelling magnetic forces RR/RF derived from and between magnets UMR and LMR with the magnetic attracting forces AFU and AFL between said magnets and said magnetically attractable material MAM, said balance of said magnetic forces mitigating a side to side rotation that could be caused by the repelling force of said magnets, as well as limiting the distance between by limiting the effect of said repelling force between said magnets. Openings SQU in magnetically attractable material MAM further provide a balance between said repelling and said attracting forces by limiting a portion of said repelling forces RF/RF (generated by said similar N poles) to pass through openings SQU, while said same magnetic force generated by said N poles of said magnets create a balancing attracting force to the magnetically attractable material MAM. The function of openings SQU as shown in this embodiment can be replaced in other embodiments by (and not limited to) variations of shape density and hole patterning within the magnetically attractable material MAM. Said variations can control and vary the strength of the magnetic forces between said components.

FIG. 1A: is a side view illustration of a rolling motion between components UMR and LMR in respect to component MAM; the axis is of maximum magnetic force AMF located between said magnetic components shifts correspondingly along the shortest distance between said magnets in relationship to said rolling motion between said magnets.

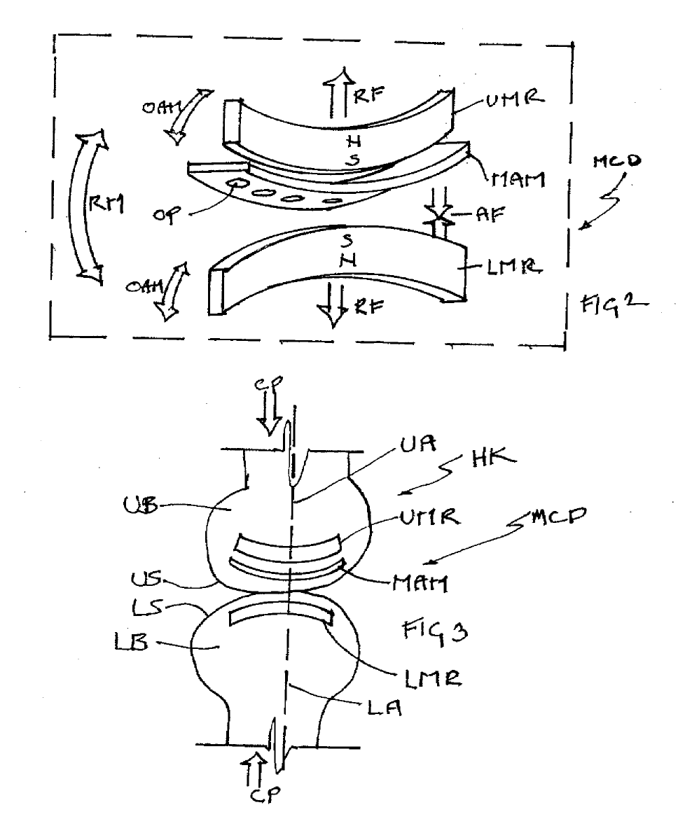

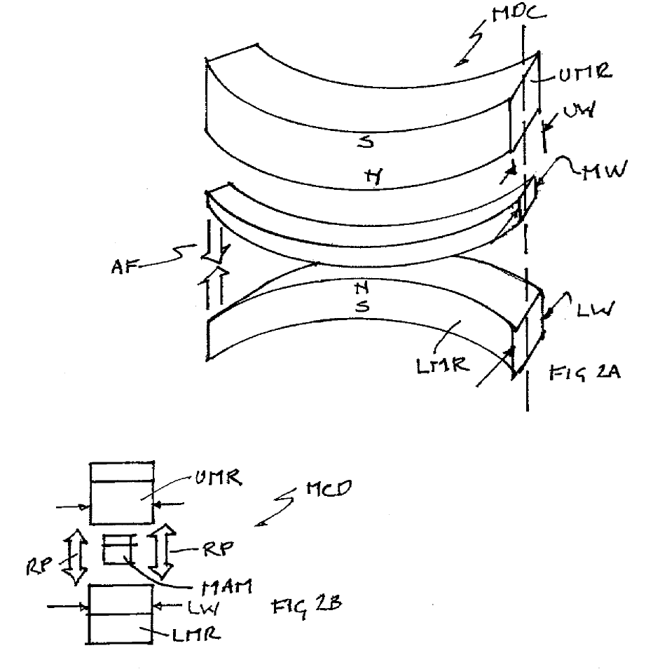

FIG. 2: Is a three dimensional diagram of a component system of a magnetic device MCD similar in function and design as the component system of the device MCD shown in FIG. 1 differing in that the magnetically attractable element MAM in this embodiment is shaped as an arc which can be somewhat concentric to one of said magnets UMR or LMR, in this embodiment said magnetically attractable element having a pattern of round openings OP. Arrow RM indicates a rotational movement between upper magnet UMR and lower magnet LMR.

FIG. 2A: is a three dimensional diagram of the components of a magnetic device MCD similar to the components of magnetic device MCD illustrated in FIG. 2 differing in that the width MW of the element of magnetically attractable material MAM is smaller than the widths UW and LW of the magnets UMR and LMR respectively. This provides a space on either side of said element MAM for repelling forces RP/RP, generated by said magnets to act upon each other wherein said same magnetic forces provide a balancing attracting force with element MAM.

FIG. 2B: is an edge view of FIG. 2A illustrating balance repelling forces RR/RP on either side of magnetically attractable material MAM.

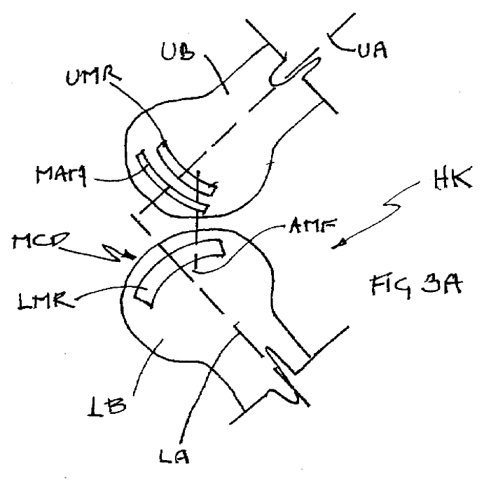

FIG. 3: Is a side view diagram of a human knee HK comprising an upper bone UB and a lower bone LB. Bones UB and LB are shown to be disposed on axis's of rotation UA and LA respectively. The component system of magnetic device MCD Such as described in FIGS. 1 thru 2B are shown as having been surgically implanted in bones UB and LB of said human knee HK, magnet UMR and magnetically attractable material MAM implanted in bone UB, magnet LMR implanted in bone LB. The balanced magnetic forces generated by said component system MCD as described in said FIGS. 1 thru 2B resist, counter and at least partially mitigate compressive forces CP/CP thus reducing wear on load bearing surfaces US and LS of bones UB and LB respectively.

FIG. 3A Illustrates bones UB and LB that when rotated on their corresponding axis's in respect to each other axis of maximum repelling force AMF shifts accordingly as described in FIG. 1A.

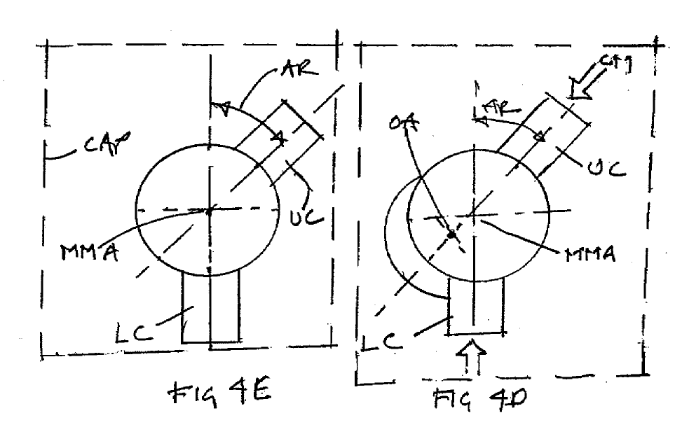

FIG. 4 is an isometric diagram of a magnetic cushioning device MCD comprising a first and second co-functional component assembly UC and LC respectively. Both said component assemblies are disposed on an intersecting plane CAP, which is coincident with the planar range of a combination of two types of motion (rotational and planar motion) that said component assemblies UC and LC can move in relationship to one another. Each said component assembly UC and LC can rotate assembly rotate about its corresponding rotational axis OA and MMA respectively. Axis OA (Axis OA is shown in FIG. 4A) and MMA being disposed substantially perpendicular to plane CAP, the shifting the point of intersection of said axis OA and MMA plane CAP represents said planar range of motion of said component assemblies. When axis OA and MMA are co-incident with each other axis MMA further represents the peak or magnetic attraction Between said component assemblies. Said range of motion is further described in FIGS. 4A, 4B, 4C, and 4D. Said first component assembly UC is comprised of a magnet M attached to an element ML (which in this embodiment is a bar of solid material) which is one of a variety of mechanical configurations that can be used to attach Magnet M to corresponding functional parts to which said magnetic cushioning device MCD can be applied. Said second component assembly LC is comprised of two plates containing magnetically attractable material MAL and MAR. Said plates MAL and MAR are attached to and held in position to each other by a “U” shaped channel SB, said “U” shaped channel SB being one of a variety of material configurations that can attach said component assembly MC onto the corresponding functional parts to which the magnetic cushioning device can be applied. One such said application is using MCD as an prosthetic medical device by implanting or attaching said first component assembly to a first bone and said second component to a second bone. Magnet M is disposed and held in a position on axial plane CAP between said plates MAL and MAR by a balanced attracting force between said magnet M and said plates MAL and MAR. Axial plane CAP is substantially central to, between, parallel and equidistant to said plates MAR and MAL. When magnetic cushioning device MCD is said to be in a resting state and is not significantly being acted upon by outside compressive forces.

FIG. 4A is a cross sectional diagram of the magnetic cushioning device MCD as illustrated and explained in FIG. 4 further showing mechanical spacers SWL and SWR which are toleranced and disposed between plates MAL and magnet M, and plate MAR and magnet M in order to maintain distance and allow for freedom of movement between said plates MAL and MAR and said magnet M. FIGS. 4B, 4C and 4D further describe the cushioning function of said device MCD as compressive forces CM/CM are applied to said device.

FIG. 4B is a is an isometric diagram of a magnetic cushioning device MCD as illustrated and described in FIGS. 4 and 4A further describing the cushioning function of said device MCD when said device is applied. As compressive forces CM/CM are substantially applied along said plane CAP, the axis of rotation of magnet M is offset to and is no longer co-incident to axis of strongest magnetic attraction along axis MMA. Arrows AD/AD indicate the distance between said axes MMA as well the attracting force between said magnet M to said plates MAL and MAR, thus resisting and at least partially mitigating compressive forces CM/CM by drawing together said axes MMA and OA. This brings said magnetic cushioning device MCD into said “resting” state. If compressive forces CM/CM are asymmetrically applied (other than 90° to each other) along said plane CAP cushioning device MCD will still tend to return to said resting state when said compressive forces CM/CM are no longer applied. This is further described in FIGS. 4D and 4E.

FIG. 4C is a side view section of FIG. 4B further illustrating the attracting force AD/AD between said axes MMA and OA in opposition to and at least partially mitigating compressive forces CL/CM.

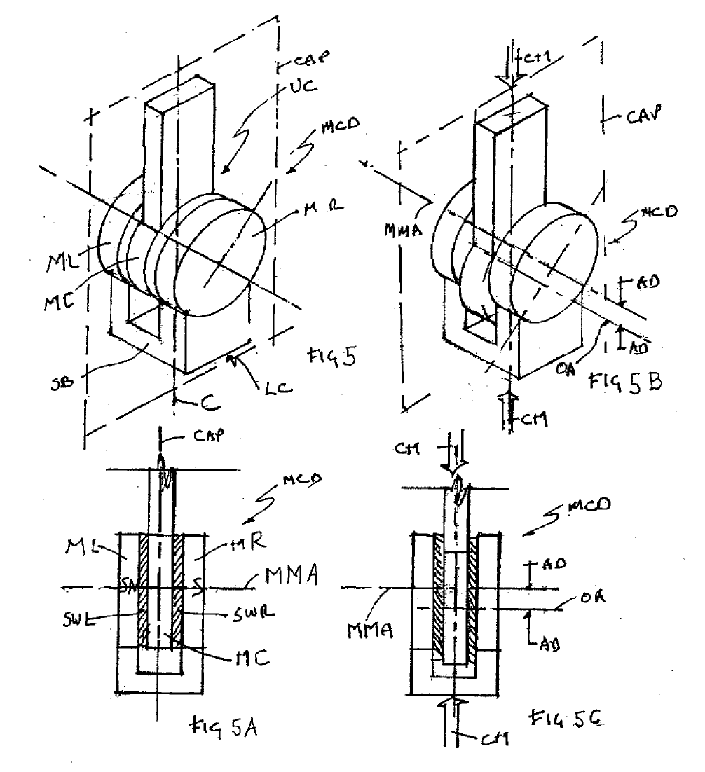

FIG. 4D is a side view elevation of the magnetic cushioning device MCD showing the rotational movement between upper component UC and lower component LC as illustrated in FIGS. 4B and 5B when said device is acted upon, by compressive force CM, CM, asymmetrically (not along a common axis), causing axes MMA and OA to be offset and non-coincident.

FIG. 4E is a side view elevation of MDC shown in FIG. 4D, having no compressive forces substantially applied. Said axis of strongest magnetic attraction MMA is coincident to the axis of rotation 6A.

FIG. 5 is an isometric diagram of a magnetic cushioning device MCD similar in structure and function to the magnetic cushioning device illustrated and described in FIG. 4, differing in that magnetically attractable plates MAR and MAL have been substituted by magnets ML and MR respectively which in this embodiment are disk shaped. Said magnetic cushioning device MCD is further described as comprising two co-functional component assemblies UC and LC. Said assembly UC, comprising magnet MC, is attached to element ML which is one of a variety of means (in this embodiment said means is a bar of solid material) that attaches magnet M to a functioning part of which magnetic cushioning device is applied. Such parts can be bones when the application is a prosthetic implant. Said component assembly MC is comprised of said two disk shaped magnets ML and MR, said magnets ML and MR are attached to and held in position with each other by a “U” shaped channel SB, said “U” shaped channel SB being one of a variety of means for attaching said component assembly MC to the functional parts to which the magnetic cushioning device is applied. Magnet MC is disposed and held in a position between said magnets ML and MR by disposing the opposite poles between and therefore the attracting forces between said magnets ML and MC and magnets MR and MC. Magnet MC is disposed substantially on axial plane CAP, which is substantially located between, and equidistant to said magnets MR and ML. Rotational axis MMA passing through is substantially perpendicular to axial plane CAP and is also the axis to the strongest (peak of magnetic flux) attracting force shared and between said magnet MC and said magnets ML and MR. when said axis MMA and OA coincident, then cushioning device MDC is said to be in a resting state. FIG. 5A is a cross sectional diagram of the magnetic cushioning device MCD as illustrated and explained in FIG. 5 further showing spacers SWL and SWR which are toleranced and disposed between said magnet ML and magnet MC, and magnet MR and magnet MC in order to maintain distance and allow for movement between said magnets. Said movement is described and illustrated in FIGS. 5B and 5C thus further describing the cushioning function of said device MCD as compressive forces CM/CM as applied to said device.

FIG. 5B is a is an isometric diagram of a magnetic cushioning device MCD as illustrated and described in FIGS. 4 and 4A and further describing the cushioning function of said device MCDC when compressive forces CM/CM are applied to said device. As compressive forces CM/CM are applied to said cushioning device MCD along said axial plane CAP, axis OA and axis MMA become offset are no longer coincident to the axis of strongest magnetic attraction MMA. Arrows AD/AD indicate the distance between said axes MMA, as well as the attracting force between said magnet MC and said magnets ML and MR, said attracting force AD/AD resist at least partially mitigates compressive forces CM/CM by pulling together said axes MMA and OA together bringing said magnetic cushioning device MCD into said “resting” state.

FIG. 5C is a side view section of FIG. 4B further illustrating the attracting force AD/AD between said axes MMA and OA in opposition to and at least partially mitigating compressive forces CM/CM.

What is claimed is:

1. A cushioning device that when implanted provides mobility and wear resisting cushioning to joints within a human body, comprising:
A first component at least in part comprising at least one magnet, said first component disposed and aligned so as to move within and sharing a planar range of motion with:
A second component at least in part comprising at least one magnet. Said second component is disposed and aligned so as to move within said predetermined planar range of motion.
The magnetic poles of said magnet of said first component, and the magnetic poles of said magnet of said second component, so disposed as to create a repelling force between said first and second components. and therefore an opposing movement away from each other.
A third component comprising a magnetically attractable material disposed between said first and second components towards which said corresponding magnets are attracted, said attracting forces balancing and controlling said same repelling force so as to maintain said movement of said first and second components within said predetermined planar degree of freedom.

2- A cushioning device as in claim 1 wherein said first and second components are arc shaped strips, the curvature of said arcs being in opposition to each other.

3- A cushioning device as in claim 1 wherein said third component is a metallic strip.

4- A cushioning device as in claim 1 wherein said third component is further comprising voids through which repelling forces between said first and second components pass.

5- A cushioning device as in claim 1 wherein said first component is implanted within a first bone and said second component is implanted in said second bone.

6- A cushioning device as in claim 5 wherein said 3^{rd} component is implanted within a first or second bone.

7- A cushioning device as in claim 1 wherein one of said components has a curved surface along which one magnetic pole is disposed, said curvature allowing said component a rolling motion in respect to said other components. Said disposition of said magnetic poles allowing a continuity of repelling and attracting forces along points of said curvatures at varying positions of said motion and between said components.

8- A cushioning device as in claim 1 wherein the width dimension of said third component in relationship to said first and second components is such as to affect said ratio and balance of the strength of the attracting forces and repelling forces between said components.

9- A cushioning device as in claim 8 wherein the width of said third component varies in dimension along its length so as to change the balance of said magnetic forces as said components move in relationship to each other within said substantially planar range of motion.

10- A cushioning device as in claim 4 wherein the ratio of open area created by said voids and the solid area changes along said third component, altering the balance of said attracting and repelling forces between said components as said components move with said substantially planar degree of freedom.

11- A magnetic cushioning comprising:
At least a first and second component assembly, said first assembly comprising a magnet attached to a component for joining said cushioning device to its intended application; said second assembly component comprising a shape composed of magnetically attractable material formed and disposed as to partially surround said magnet of said first component assembly, said shape attached to a component for joining said cushioning device to its intended application;
Said first and second component assemblies so disposed within an axial plane of movement and each having a rotational axis that is perpendicular to said axial plane. The peak of attracting forces (magnetic flux) between said magnets of said first component assembly and said surrounding shape of magnetically attracting material of said second component assembly, extends along and exists when said rotational axes are coincident and common to each other;
Said attracting force between said first and second component assemblies counteracting compressive forces of the cushioning device.

12- A magnetic cushioning device as in claim 11 wherein said magnet of said first component assembly is planar, the surfaces of which being substantially parallel to said axial plane. Said shape of magnetically attractive material is in the form of a “U” channel, said legs of said U channel being disposed on either side of said axial plane and substantially parallel to said axial plane and said magnet.

13- A magnetic cushioning device as in claim 11 wherein said surrounding shape of said second component assembly is formed as a “U” shaped channel, with the sides of said channel being magnets. The faces of said magnets are substantially parallel to said axial plane and the faces of said magnet of said first component assembly, the poles of said second component magnets disposed and opposite that face the magnet of the first component.

14- A magnetic cushion device as in claim 13 wherein said first component assembly comprises a plate of magnetically attractable material.

15- A magnetic cushioning device as in claim 12 wherein said U-shaped channel is a horseshoe type magnet.

16- A magnetic cushioning device as in claim 1 where in said third component is comprised of a material that is not magnetically attractable shaped and disposed between said 1^{st} and 2^{nd} components so as to intercept and obstruct portions of said magnetic repelling force between said 1^{st} and 2^{nd} components.
